(19) United States
(12) Patent Application Publication
(10) Pub. No.: US 2007/0286807 A1
(43) Pub. Date: Dec. 13, 2007

(54) Method Of Inducing Or Modulating Immune Response

(76) Inventor: Susan Marie Metcalfe, Cambridge (GB)

Correspondence Address: SUTHERLAND ASBILL & BRENNAN LLP, 999 PEACHTREE STREET, N.E., ATLANTA, GA, 30309, US

(21) Appl. No.: 10/587,995

(22) PCT Filed: Jan. 31, 2005
(86) PCT No.: PCT/EP05/00934

§ 371 Date: Jul. 27, 2007

(30) Foreign Application Priority Data
Jan. 29, 2004 (GB) 0402051.7

(51) Int. Cl. A61K 31/711; A61K 38/16; A61K 49/00; A61P 43/00; C07H 21/04; C07K 14/435; C12N 15/00; C12N 5/06; C12Q 1/68; G01N 33/50; G01N 33/53

(52) U.S. Cl. 424/9.2; 435/320.1; 435/375; 435/6; 435/7.1; 436/501; 436/94; 514/12; 514/44; 530/350; 536/23.1

(57) ABSTRACT

The use of axotrophin, also known as MARCH VII to induce or regulate immune response to an antigen whether foreign or self, suitably in a vertebrate, for example a mammal is disclosed. Isolated axotrophin and nucleotides and polypeptides encoded by or derived from axotrophin, compositions containing one or more thereof and assay methods are provided as further aspects of the invention.

The present invention relates to the use of a polynucleotide, polypeptide and proteins encoded by or derived from such polynucleotide, along with uses for the polynucleotide, polypeptide and proteins and to a method of inducing or modulating immune response to an antigen and further relates to a method of determining the immune status of an individual with respect to a given antigen. In particular, the invention relates to the use of axotrophin, also known as MARCH VII to induce or regulate immune response to an antigen whether foreign or self, suitably in a vertebrate, for example a mammal. The invention also provides isolated axotrophin and nucleotides and polypeptides encoded by or derived from axotrophin, compositions containing one or more thereof and assay methods.

As used herein, reference to axotrophin includes a reference to a polynucleotide or polypeptide sequence having at least 75% and preferably at least 90% sequence identity to an identifying sequence of axotrophin. The finding that axotrophin plays a significant role in the immune response of an individual enables its use in numerous applications in a variety of techniques known to those skilled in the art of molecular biology, such as use as hybridization probes, use as primers for PCR, use in an array, use in computer-readable media, use in sequencing full-length genes, use in the recombinant production of protein, and use in the generation of anti-sense DNA or RNA, their chemical analogs and the like.

Identified polynucleotide and polypeptide sequences have numerous applications in, for example, diagnostics, forensics, gene mapping, identification of mutations responsible for genetic disorders or other traits, to assess biodiversity, use as primers in expression assays and to produce many other types of data and products dependent on DNA and amino acid sequences. Axotrophin is known and details of the axotrophin gene may be found in the GenBank database and elsewhere under various Accession Numbers including AK022973 and NM_{—}022826.2 (human) and AF155739 and NM_{—}020575 (murine). Human axotrophin protein sequence may be found under Accession Number NP_{—}073737.1 and murine axotrophin protein under NP_{—}065600.1. These sequences are set out below as Sequence Idenitification Numbers 001 to 004 respectively. Axotrophin is one of 216 genes identified as being enriched in mouse embryonic, neural and hematopoietic stem cells as disclosed in Science, Vol 298, 597-600 18 Oct. 2002 and is said (in Table 1) to participate in signaling and the ubiquitin pathway. Genes & Development 15:2660-2674 published in 2001 discloses that mouse protein axotrophin has a RING-CH domain and is required for normal brain development and that disruption of the axotrophin gene may result in neural degeneration and callosal agenesis. There would appear to be little else known about the function of axotrophin from the published literature.

The present inventor has now found that axotrophin induces or regulates immune response to an antigen at the genomic, mRNA and/or protein level. It is believed regulation may be manipulated through antisense DNA or RNA or binding molecules. Additionally, axotrophin has been found to regulate T lymphocyte cell proliferation and to regulate release of leukemia inhibitory factor (LIF) for example from activated T lymphocyte cells as set out in the Examples below. WO 03/052424 discloses that c-kit (CD117), STAT3, stem cell factor (SCF) and LIF are elevated in tolerant immune responses and that these may be used in modulating immune response generated to an antigen. A LIF murine sequence is available at SWISSPROT P09056. A human sequence is available at SWISSPROT P15018.

The invention provides the use of axotrophin or a polypeptide or polynucleotide encoded by or derived from axotrophin to induce or to regulate directly or indirectly the immune response to an antigen, whether a “foreign” antigen (for example allogeneic, xenogeneic, procaryotic, viral or synthetic) or autologous (“self”) antigen.

Manipulation of the immune response may be in ex vivo, in vivo or in vitro cell population.

Any reference to “regulation” of the immune response in relation to this invention includes regulating phenotypic development and maintenance of cell populations that regulate immunity to a given antigen.

Reference herein to materials “derived from” axotrophin includes, by way of example, anti-sense sequences including RNAi, whether single or multiple stranded, and small molecules binding to polypeptides or polynucleotides of axotrophin, including antibody especially monoclonal antibody. Reference to materials derived “directly or indirectly” from axotrophin includes any such polynucleotides or small molecules.

Reference herein to “polypeptide” includes protein and especially mature protein.

The invention also provides the use of axotrophin or a polypeptide or polynucleotide encoded by or derived from axotrophin in the manufacture of a medicament to induce or to regulate directly or indirectly the immune response of a vertebrate to an antigen, whether a “foreign” antigen (for example allogeneic, xenogeneic, prokaryotic, viral or synthetic) or autologous (“self”) antigen.

The medicament produced according to the invention is suitable for treating an individual to reduce rejection of transplanted tissue, cells or organ.

The invention further provides for use of axotrophin or a polynucleotide encoded by or derived from axotrophin to regulate expression of LIF. LIF may induce or regulate directly or indirectly the immune response of a vertebrate to an antigen, whether a “foreign” antigen (for example allogeneic, xenogeneic, procaryotic, viral or synthetic) or autologous (“sew”) antigen.

Suitably, use of polypeptide or polynucleotide encoded by or derived from axotrophin allows cancerous immune cells that are sensitive to LIF to be targeted ex vivo or in vivo,

Without wishing to be bound by any theory, it is believed that axotrophin also regulates the expression of Foxp3 and SOCS3 at the genomic and/or protein level and that this plays a role in T cell regulation.

The invention provides in a further embodiment for use of axotrophin or a polypeptide or polynucleotide encoded by or derived from axotrophin to induce or regulate T cell proliferation in a cell population in an in vivo, ex vivo or in vitro environment The T cells are preferably T lymphocyte cells.

Advantageously, the present invention may be used to guide the immune response of a vertebrate for example a mammal to accept a transplanted organ, tissue, cell, gene or gene product, artificial substance, or any other agent utilized within the body, for example for a therapeutic purpose. The invention is especially applicable in the use of stem cells In therapy or otherwise.

The immune suppressive activity of axotrophin may be used to protect introduced biological materials from immune attack, for example in transplantation of cells, to treat diseases including neurodegenerative diseases, tissues for grafting or example bone marrow, skin, cartilage, bone, tendons, muscle including cardiac muscle, blood vessels, cornea, neural cells, gastrointestinal cells and others and organs for transplantation including kidney, liver, pancreas including the Islet cells, heart and lung.

Suitably, expression of the encoded or derived from axotrophin polypeptide or regulatory polypeptide or polynucleotide sequences that influence axotrophin activity may be modified in the host immune cells ex vivo to bias the immune response to accept the introduced biological materials, Alternatively, or additionally, expression of axotrophin within the biological materials may be modulated ex vivo to carry immunomodulatory properties when introduced in vivo.

Axotrophin may be employed in the treatment of immune disorders including severe combined immunodeficiency (SCID) by regulating, up or down, T lymphocytes as well as effecting the cytolytic activity of NK cells and other cell populations. These immune deficiencies may be genetic or be caused by viral (for example, HIV) as well as bacterial or fungal infections, or may result from autoimmune disorders, More specifically, infectious diseases caused by viral, bacterial, fungal or other infection may be treatable using a protein or polynucleotideencoded by or derived from axotrophin including infections by HIV, hepatitis viruses, herpes viruses, mycobacteria, Leishmania spp., malaria spp. and various fungal infections such as candidiasis as well as where a boost to the immune system generally may be desirable, for example in the treatment of cancer.

Autoimmune disorders which may be treated using a protein or polynucleotide encoded by or derived from axotrophin include, for example, connective tissue disease, multiple sclerosis, systemic lupus erythematosus, rheumatoid arthritis, autoimmune pulmonary inflammation, Guillain-Barre syndrome, autoimmune thyroiditis, insulin dependent diabetes mellitis, myasthenia gravis, graft-versus-host disease and autoimmune inflammatory eye disease. Such a protein (or antagonists thereof, including antibodies) of the present invention may also to be useful in the treatment of allergic reactions and conditions (for example, anaphylaxis, serum sickness, drug reactions, food allergies, insect venom allergies, mastocytosis, allergic rhinitis, hypersensitivity pneumonitis, urticaria, angioedema, eczema, atopic dermatitis, allergic contact dermatitis, erythema multiforme, Stevens-Johnson syndrome, allergic conjunctivitis, atopic keratoconjunctivitis, venereal keratoconjunctivitis, giant papillary conjunctivitis and contact allergies), such as asthma (particularly allergic asthma) or other respiratory problems.

In using axotrophin, down regulation may be in the form of inhibiting or blocking an immune response already in progress or may involve preventing the induction of an immune response.

The use of axotrophin in down regulating or preventing one or more functions during the immune response for example in reducing interferon gamma release, may be useful in situations of tissue, skin and organ transplantation and in graft-versus-host disease (GVHD). Up regulating aggressive immune responses by down modulation of axotrophin or a polynucleotide or polypeptide encoded by or derived from axotrophin is also useful. Upregulation of immune responses may be in the form of enhancing an existing immune response or eliciting an initial immune response. For example, enhancing an immune response may be useful in cases of viral infection, including systemic viral diseases such as influenza and the common cold. Regulation of axotrophin suitably facilitates a T cell-mediated immune response against tumour cells.

A polypeptide of axotrophin may be involved in regulating in chemotactic or chemokinetic activity for mammalian cells, including, for example, monocytes, fibroblasts, neutrophils, T-cells, mast cells, eosinophils, epithelial and/or endothelial cells. The invention provides chemotactic or chemokinetic compositions for example proteins, antibodies, binding partners, or modulators containing axotrophin or polynucleotides or polypeptides encoded by or derived from axotrophin, provide particular advantages in treatment of wounds and other trauma to tissues, as well as in treatment of localized infections. For example, attraction of lymphocytes, monocytes or neutrophils to tumors or sites of infection may result in improved immune responses against the tumor or infecting agent.

The invention may also suitably be used to guide the immune system to allow for acceptance of, or at least reduced aggressive response to, an antigen associated with an autoimmune disease or disorder, whether eliciting the innate or adaptive immune response during the auto-immune reaction.

Further, the invention may be used to guide the immune response to reject an organ, tissue, cell, pathogen such as a prokaryote, yeast or fungus, parasite or virus, a gene or gene product, an artificial substance, or any other agent that may invade or be taken into the body, or be generated within the body, wherein that agent is unwanted, diseased (for example neoplastic tissue or infected tissue), or otherwise deleterious to the host patient.

The invention also may be used to enhance the degree of immune response against antigen following vaccination, especially in cases where current vaccination procedures are off limited success in generating a protective immune rejection response against biological agents, including for example those associated with germ warfare

An especially advantageous aspect of the invention is the specificity of response generated on activation by a specific antigen. The immune response may be guided to tolerance or aggression by signal pathway modulation in vivo. On challenge with an antigen, responsive cells may be guided towards tolerance or aggression in accordance with various aspects of the invention and non-responsive cells remain unaffected by the regulatory adaptation. The target antigen itself triggers responsive cells or responsive cell populations: cells capable of responding only to other antigens are not triggered, and are therefore not receptive to guiding towards tolerance or aggression towards the relevant antigen at that time. As an alternative or supplement, Immune cells may be guided to regulatory tolerance, or aggression ex vivo. Immune cells, for example of blood and/or spleen, may be removed, treated with antigen and guided to tolerance or aggression, before being returned to the individual.

As used herein, the term “antigen” has the meaning generally understood in the ar and includes any naturally occurring, recombinant or synthetic product such as a polypeptide, which may be glycosylated. The term antigen also includes complexes of protein carriers and non-protein molecules such as steroids, carbohydrates or polynucleotides.

Antigen is also used herein to refer to any substance which comprises a plurality of antigens and opitopes, for example a cell or tissue, organ, implant, indeed any substance to which an immune response can be mounted by the immune system of a vertebrate, for example a mammal.

The antigen may be an antigen of a pathogenic organism associated with human or animal disease. Organisms which cause animal disease include for example foot and mouth disease virus, Newcastle disease virus, rabies virus and Salmonella species. Organisms which cause human disease include for example bacteria such as Salmonella species including S. typhimurium and S. typhi, Staphylococcus such as S. aureus, Pertussis, Vibrio cholera, pathogenic E. coli, Mycobacteria species such as M. tuberculosis and M.paratuberculosis. Viral organisms include for example HIV-1 or HIV-2 (which include the viral antigens gpl60/120), HBV (which includes surface or core antigens), HAV, HCV, HPV (for example HPV-16), HSV-1 or-2, Epstein Barr virus (EBS), neurotropic virus, adenovirus, cytomegalovirus, polio myelitis virus, and measles virus.

Small pox and anthrax are also pathogens of interest and which may be subject to the present invention. Eukaryotic pathogens include yeast, such as C. albicans, aspergillus, schistosomes, protozoans, amoeba, plasmodia, including for malaria, toxoplasma, giardia and leishmania.

The antigen may also be a tumour associated antigen. Such antigens include GEA, alpha fetal protein (AFP), neu/HER2, polymorphic endothelia mucin (PEM), N-CAM and Lewis Y.

The antigen may be an abnormally expressed antigen, such as p53 or virally-modified antigen.

Antigens such as those mentioned above may be obtained in the form of proteins purified from cultures of the organism, or more preferably by recombinant production of the desired antigen. Antigens may also be produced by chemical synthesis, for example employing an automated peptide synthesiser such as are commercially available.

Instead of wild-type polypeptide, an appropriate fragment may be used provided the desired activity is retained. The skilled person is readily able to make changes to amino acid sequence of any polypeptide in a conservative manner, for example without abolishing function.

A further aspect of the present invention provides a method of modulating an immune response to an antigen in an individual, the method including provision in the individual of axotrophin or a polypeptide or polynucleotide encoded by or derived from axotrophin.

Such provision may be by administration of the polypeptide or polypeptides, or may be by administration of polynucleotide encoding the polypeptide or polypeptides. A further approach comprises administration of a substance that upregulates expression of the polypeptide or polypeptides, for example by binding the promoter or other regulatory element of the relevant gene.

The present invention also provides for a method of modulating an immune response of an individual to an antigen, the method comprising administering a substance that affects activity of axotrophin in the individual,

The amount of polypeptides expressed directly or indirectly by axotrophin in the individual may be modulated either upwards, so that activity is increased or augmented, or downwards, so that activity is decreased or reduced. Increased activity is associated with a promotion of immune tolerance, while decreased activity is associated with a promotion of immune response against the antigen, that is an aggressive response.

Thus, in accordance with the present invention there is provided a method of manipulating the response of the immune system to a given antigen, for example increasing tolerance of the immune system of an individual to an antigen, the method comprising administering to the individual axotrophin or a polypeptide or polynucleotide encoded by or derived from axotrophin or a substance that enhances the amount or activity of polypeptide expressed directly or indirectly by axotrophin.

Further, in accordance with the present invention there is provided a method of potentiating or increasing the aggressive response of the immune system of an individual against an antigen, the method comprising administering to the individual a substance that decreases the amount or activity of a polypeptide expressed directly or indirectly by axotrophin.

A substance may decrease the amount or activity of polypeptide expressed directly or indirectly by axotrophin by binding or otherwise interacting with it. Such a substance may be for example an antibody molecule with appropriate binding specificity, or other peptidyl or non-peptidyl molecule that binds the polypeptide. Production of the polypeptide, may be reduced by for example down-regulating promoter function of the relevant gene or by targeting encoding mRNA to reduce translation (for example by antisense or dsRNA inhibition, RNAi, or ribozyme digestion) or by means of a substance that promotes degradation of the polypeptide, for example using ubiquitination.

A substance may increase activity of polypeptide expressed directly or indirectly by axotrophin by means of binding, for instance by binding to a promoter or enhancer region of an encoding polynucleotide sequence to increase promoter function.

A further aspect of the invention provides a method of enhancing an aggressive immune response against an antigen in an individual, or of providing an enhanced aggressive immune response or reduced aggressive immune response, or of promoting tolerance in an individual, the method comprising administering to the individual a composition comprising the antigen or polynucleotide encoding the antigen and administering a composition which comprises a polypeptide expressed directly or indirectly by axotrophin or a substance that alters the amount or activity of such a polypeptide in an individual.

Two or more compositions may be provided as a combined preparation for simultaneous or sequential administration.

The level of materials produced on expression of axotrophin, for example LIF may be altered, for example via encoding polynucleotide, or by alteration of endogenous expression levels, or by alteration of polypeptide activity, for example by means of a small molecule or other active agent, so as to modulate the presence or degree of tolerance or aggression that the immune system of an individual shows to an antigen of interest. The present invention may be used in a variety of contexts, including conditioning of the immune system with respect to a planned transplant, to potential challenge with a pathogen or other foreign body, to transformed cells of the host, for example cancer cells or virally-infected cells, and in an autoimmune disorder.

An aggressive immune response modulated or affected in accordance with the present invention may be an inappropriate immune response, for example in an autoimmune disease, or an appropriate immune response, for example in response to a pathogen.

Axotrophin has been found to provide regulation of the immune response in a vertebrate for example a mammal including human. Suitably the response is a tolerogenic immune response to an antigen in the vertebrate.

In a further embodiment, the present invention provides for use of axotrophin or a polypeptide or polynucleotide encoded by or derived from axotrophin for assaying immune status. Axotrophin or a polypeptide or polynucleotide encoded by or derived from axotrophin is suitable for useful in clinical medicine or veterinary medicine.

The invention also provides a method for determining immune status of an individual, the method comprising determining the level of expression of axotrophin or a polypeptide or polynucleotide encoded by or derived from axotrophin in a test sample comprising tissue, cells and/or bodily fluid removed or obtained from the individual and comparing the level for the test sample with that of a control sample, wherein a level in the test sample greater than that of the control sample is indicative that the immune status in the individual comprises a tolerant immune response, or wherein a level in the test sample lower than that of the control sample is indicative that the immune status in the individual comprises an aggressive immune response.

An assay of immune status may be used to assess immune status of an individual in relation to immune response to a pathogen, immune response to a diseased tissue such as a tumour, tolerance to a transplanted tissue, cell or other material (for example to indicate a status of tolerance to an organ allograft or xenograft when it is desired to reduce or remove immunosuppressive therapy to the recipient). Thus, such an assay may be used in a diagnostic context, to determine the status of the immune system of an individual. It may be used to assess the benefit or success of ongoing treatment.

The method is particularly beneficial for determining immune status of an individual having a tissue or cell transplant and optionally is undergoing therapy Suitably, the level is determined for a test sample comprising peripheral blood. Reference herein to an “individual” includes animal as well as human.

A further aspect provides for use of axotrophin or a polypeptide or polynucleotide encoded by or derived from axotrophin or a substance that alters amount or activity thereof in an individual as disclosed, in the manufacture of a medicament to boost or reduce an aggressive immune response in an individual against an antigen or to alter tolerance of the immune system to an antigen, or for use in any method of treatment as set out herein. Such a medicament is generally for administration for treatment or prevention of a disease or disorder associated with the antigen, whether the antigen be of a pathogen, disease cell such as a tumour, or a material to be transplanted, such as an organ, tissue or cell.

Generally, such a substance according to the present Invention is provided in an isolated and/or purified form, that is substantially pure. In a preferred embodiment, the substance is in a composition where it suitably represents at least 80% active ingredient, preferably at least 90%, more preferably at least 95% and especially at least 98% by weight of the composition.

A polypeptide encoded by or derived from axotrophin or a peptidyl substance that affects the activity or amount such a polypeptide, for example by binding with it (such as an antibody molecule) or by binding with a promoter element that affects the polypeptide production by expression from the encoding gene, or other polypeptide that may be used in any aspect or embodiment of the present invention, may be produced by recombinant expression.

A substance to be given to an individual in accordance with an embodiment of the present invention may be administered in a “prophylactically effective amount” or a “therapeutically effective amount” as desired. A prophylactic effect may be sufficient to potentiate or reduce an aggressive immune response of an individual to a subsequent challenge with antigen (depending on whether an aggressive immune response against antigen of a tolerigenic response is desired). Most preferably the effect is sufficient to prevent the individual from suffering one or more clinical symptoms as a result of subsequent challenge with antigen. A therapeutic effect is sufficient to potentiate or reduce an aggressive immune response of an individual to pre-existing reaction, preferably sufficient to antagonise the reaction, wholly or partially, for example in an autoimmune disorder or in transplant rejection. Most preferably the effect is sufficient to ameliorate one or more clinical symptoms. The actual amount administered, and rate and time-course of administration, will depend on the nature and severity of what is being treated. Prescription of treatment, for example decisions on dosage etc, is within the responsibility of general practitioners and other medical doctors, and typically takes account of the disorder to be treated, the condition of the individual patient, the site of delivery, the method of administration and other factors known to practitioners, Examples of the techniques and protocols mentioned above can be found in Remington's Pharmaceutical Sciences, 16th edition, Osol, A. (ed), 1980.

The invention also provides a ribozyme having specificity for a polynucleotide of the invention based upon the nucleotide sequence of axotrophin.

In addition, the invention encompasses methods for the manufacture of a medicament for treating conditions of or related to the immune system comprising administering a compound or other substance that modulates the overall activity of axotrophin or a polypeptide or polynucleotide encoded by or derived from axotrophin. Compounds and other substances can effect such modulation either on the level of target gene/protein expression or target protein activity.

The invention in a further aspect provides isolated axotrophin a polynucleotide or a polypeptide encoded by or derived from axotrophin, including recombinant DNA molecules, cloned genes or degenerate variants thereof, especially naturally occurring variants such as splice variants, allelic variants, antisense polynucleotide molecules, and antibodies that specifically recognise one or more epitopes present on such polypeptides, as well as hybridomas producing such antibodies.

The polynucleotide sequences of the present invention also include a segment of axotrophin that uniquely identifies or represents the sequence information of axotrophin. Isolated polynucleotide sequences may be produced by cloning the appropriate polynucleotide sequence and expressing it in a vector according to methods known in the art.

The polynucleotides of the present invention also include a polynucleotide that hybridizes under stringent hybridization conditions to (a) the complement of axotrophin; (b) a polynucleotide nucleotide sequence encoding axotrophin; (c) a polynucleotide which is an allelic variant of axotrophin; (d) a polynucleotide which encodes a species homolog (for example orthologs) encoded by or derived from axotrophin or (e) a polynucleotide that encodes a polypeptide comprising a specific domain or truncation of any of the polypeptides encoded by or derived from axotrophin.

As a means of providing the immune response, delivery of a functional gene encoding polypeptides encoded by or derived from axotrophin to appropriate cells is suitably effected ex vivo, in situ, or in vivo suitably by the use of vectors, and more particularly viral vectors (for example, adenovirus, adeno-associated virus, or a retrovirus), or ex vivo by use of physical DNA transfer methods (for example, liposomes or chemical treatments). Naked DNA or RNA may be used for expression of an encoded gene product in vivo. Naked DNA may be delivered using direct injection or by use of gene-guns (Yang etal., 1990) or any other suitable technique, such as topically for example for treatment of psoriasis. Cells transformed or transfected or otherwise genetically engineered to contain axotrophin or a polynucleotide encoded or derived from thereby or to express axotrophin polypeptide may be employed to deliver the functional material.

In a further aspect, the invention provides a vector for the expression of axotrophin, a polynucleotide sequence or a polypeptide encoded by or derived from axotrophin, the vector containing axotrophin or a polynucleotide sequence encoding axotrophin, for example a polynucleotide sequence complementary thereto or the reverse thereof a promoter sequence and a termination sequence.

Viral vectors may be used to deliver axotrophin or a polynucleotide encoded by it for production, suitably in vivo. Axotrophin or a polynucleotide encoded by axotrophin which encodes a polypeptide or other peptidyl molecule for use according to the present invention may be used in a method of gene therapy. This requires use of suitable regulatory elements for expression and a suitable vector for deliver of the expression unit (coding sequence and regulator elements) to host cells in vivo. A variety of vectors, both viral vectors and plasmid vectors, are known in the art, see for example U.S. Pat. No. 5,252,479 and WO 93/07282 and countess other publications. In particular, a number of viruses have been used as gene transfer vectors, including papovaviruses, such as SV40, vaccinia virus, herpes viruses, including HSV and EBV, and retroviruses. Many gene therapy protocols in the prior art have used disabled murine retroviruses. A variety of adenovirus and adeno-associated viral vectors have been developed. Alternatives to viral vectors include transfer mediated by liposomes and direct DNA uptake and receptor-mediated DNA transfer.

Expression of polynucleotides or polypeptides encoded by or derived from axotrophin is suitably under the control of inducible regulatory elements, in which case the regulatory sequences of the endogenous gene may be replaced by homologous recombination. Gene targeting may be used to replace a gene's existing regulatory region with a regulatory sequence isolated from a different gene or a novel regulatory sequence synthesized by genetic engineering methods. Such regulatory sequences may be comprised of promoters, enhancers, scaffold-attachment regions, negative regulatory elements, transcriptional initiation sites, regulatory protein binding sites or combinations of said sequences. Alternatively, sequences which affect the structure or stability of the RNA or protein produced may be replaced, removed, added, or otherwise modified by targeting. These sequences include polyadenylation signals, mRNA stability elements, splice sites, leader sequences for enhancing or modifying transport or secretion properties of the protein, or other sequences which alter or improve the function or stability of protein or RNA molecules.

Other methods inhibiting expression of a polypeptide include the introduction of antisense molecules to the polynucleotides of the present invention, their complements, their transcribed RNA sequences, or translated products of RNA by methods known in the art. Further, the polypeptides of the present invention can be inhibited by using targeted deletion methods, or the insertion of a negative regulatory element such as a silencer, which is tissue specific. “Gene silencing” technology is disclosed by Fire et al in EP-A-1042462 and Nature Vol 391 pp 806 to 811, “Potent and specific genetic interference by double stranded RNA in C elegans”.

The term “isolated” as used herein refers to a polynucleotide or polypeptide separated from at least one other component (for example, polynucleotide or polypeptide) present with the polynucleotide or polypeptide in its natural source. In one embodiment, the polynucleotide or polypeptide is found in the presence of (if anything) only a solvent, buffer, ion, or other component normally present in a solution of the same. The terms “isolated” and “purified” do not encompass polynucleotides or polypeptides present in their natural source.

The term “degenerative variant” as used herein includes nucleotide sequences that differ from the sequence according to the invention but due to the degeneracy encode an identical polypeptide sequence or a sequence having at least 75% and preferably at least 90% sequence identity thereto.

A collection of sequence information for axotrophin or identifying information for it can be provided on a polynucleotide array. In one embodiment, segments of sequence information are provided on a polynucleotide array to detect the polynucleotide that contains axotrophin or an axotrophin segment. The array can be designed to detect full-match or mismatch to axotrophin. The collection can also be provided in a computer-readable format.

The invention further provides cells genetically engineered to contain axotrophin or a vector according to the invention as described herein. Suitably the cells according to the invention, preferably host cells, have been transformed or transfected with axotrophin or another polynucleotide of the invention to express axotrophin or a polynucleotide or polypeptide sequence encoded by or derived from axotrophin. Known transformation, transfection or infection methods may be employed.

Systems for cloning and expression of a polynucleotide or polypeptide in a variety of different cells are known. Suitable host cells include bacteria, eukaryotic cells such as mammalian and yeast, and baculovirus systems. Mammalian cell lines available in the art for expression of a heterologous polypeptide include Chinese hamster ovary cells, HeLa cells, baby hamster kidney cells, COS cells and many others. A common, preferred bacterial host is E. coli.

A still further aspect provides a method which includes introducing the polynucleotide into a host cell. The introduction, which may (particularly for in vitro introduction) be generally referred to without limitation as a transformation, may employ any available technique. For eukaryotic cells, suitable techniques may include calcium phosphate transfection, DEAE-Dextran, electroporation, liposome-mediated transfection and transduction using retrovirus or other virus, for example vaccinia or, for insect cells, baculovirus. For bacterial cells, suitable techniques may include calcium chloride transformation, electroporation and transfection using bacteriophage. As an alternative, direct injection of the polynucleotide could be employed. Marker genes such as antibiotic resistance or sensitivity genes may be used in identifying clones containing polynucleotide of interest, as is well known in the art.

The introduction may be followed by causing or allowing expression from the polynucleotide, for example by culturing host cells (which may include cells actually transformed although more likely the cells will be descendants of the transformed cells) under conditions for expression of the gene, so that the encoded peptide or polypeptide is produced. If the peptide or polypeptide is expressed coupled to an appropriate signal leader peptide it may be secreted from the cell into the culture medium. Following production by expression, a peptide or polypeptide may be isolated and/or purified from the host cell and/or culture medium, as the case may be, and subsequently used as desired, for example in the formulation of a composition

Suitably the polynucleotides of axotrophin expressed in cells in vivo are in operative association with a regulatory sequence heterologous to the host cell which drives expression of the polynucleotides in the cell. These methods can be used to increase or decrease the expression of the polynucleotides of the present invention. The invention also relates to methods for producing axotrophin polypeptide comprising growing a culture of cells of the invention in a suitable culture medium under conditions permitting expression of the desired polypeptide, and purifying the polypeptide from the culture or from the host cells. Preferred embodiments include those in which the protein produced by such process is a mature form of the protein and any other polypeptides that retain any functional activity of the mature protein. In a preferred embodiment, a polypeptide encoded by or derived from axotrophin is used to generate an antibody that specifically binds the polypeptide. Such antibodies, particularly monoclonal antibodies, are useful for detecting or quantitating the polypeptide in tissue especially for immune diagnostic purposes. Polypeptides of the invention may be produced in whole or part by recombinant means but may be chemically synthesized

Such a method may comprise bringing a population of antibody molecules into contact with axotrophin or a polynucleotide or polypeptide encoded by or derived from axotrophin and selecting one or more antibody molecules of the population able to bind and/or affect the activity of the polypeptide or polynucleotide.

Antibody molecules may routinely be obtained using technologies such as phage display, by-passing direct involvement of an animal's immune system. Instead of or as well as immunising an animal, a method of obtaining antibody molecules as disclosed may involve displaying the population of antibody molecules on the surface of bacteriophage particles, each particle containing polynucleotide encoding the antibody molecule displayed on its surface, Polynucleotide may be taken from a bacteriophage particle displaying an antibody molecule able to bind a peptide or peptides of interest, for manipulation and/or use in production of the encoded antibody molecule or a derivative thereof (for example a fusion protein, a molecule including a constant region or other amino acids, and so on). Instead of using bacteriophage for display (as for example in WO92/01047), ribosomes or polysomes may be used, for example as disclosed in U.S. Pat. No. 5,543,768, U.S. Pat. No. 5,658,754, WO95/11922.

A peptide or peptides may be administered to a non-human mammal to bring them into contact with a population of antibody molecules produced by the mammal's immune system, then one or more antibody molecules able to bind the peptide or peptides may be taken from the mammal, or cells producing such antibody molecules may be taken from the mammal. The mammal may be sacrificed.

If cells are taken from the mammal, such cells may be used to produce the desired antibody molecules, or descendants or derivative cell lines may be used. Such descendants or derivatives in particular may include hybridoma cells.

Antibody molecules may be provided in isolated form, either individually or in a mixture. A plurality of antibody molecules may be provided in isolated form.

Preferred antibodies according to the invention are isolated, in the sense of being free from contaminants such as antibodies able to bind other polypeptides and/or free of serum components. Monoclonal antibodies are preferred for some purposes, though polyclonal antibodies are within the scope of the present invention.

Antibodies useful in accordance with the present invention may be modified in a number of ways. Indeed the term “antibody molecule” should be construed as covering antibody fragments and derivatives comprising an antibody antigen-binding domain enabling it to bind an antigen or epitope. Example antibody fragments, capable of binding an antigen or other binding partner are the Fab fragment consisting of the VL, VH, Cl and CH1 domains; the Fd fragment consisting of the VH and CH1 domains; the Fv fragment consisting of the VL and VH domains of a single arm of an antibody; the dAb fragment which consists of a VH domain; isolated CDR regions and F(ab′) 2 fragments, a bivalent fragment including two Fab fragments linked by a disulphide bridge at the hinge region. Single chain Fv fragments are also included.

Cells may be cultured ex vivo in the presence of proteins or polynucleotides encoded by or derived from axotrophin in order to generate a desired immune response for example immunosuppression for subsequent reintroduction in viva to allow introduction of immunogenic biological material. In other uses, prevention of the expression or inhibiting the activity of axotrophin may be desirable so as to augment aggressive immune activity against antigens. Antisense therapy or gene therapy may suitably be employed to negatively regulate the expression of polypeptides or polynucleotides encoded by or derived from axotrophin.

Modification of cells or tissues to permit, increase or decrease expression of endogenous axotrophin polypeptide to provide increased polypeptide expression by replacing in whole or part the naturally occurring promoter with a heterologous promoter so that the cells express the protein at higher levels or show induced expression in response to pharmaceutical compounds.

In a further aspect, the invention provides for manipulating, for example enhancing production of autologous or other stem cells or precursor cells and/or immune cells ex vivo by introduction to the cell of axotrophin or a polynucleotide or polypeptide encoded by or derived from axotrophin, The cells are manipulated prior to in vivo delivery for therapeutic purpose, particularly for regulating the immune response.

In a preferred embodiment, lymphocytes from an individual may be cultured ex vivo in the presence of one or more specific differentiation factors (for example target antigen for a given T cell receptor (“TCR”) and the response to that antigen adapted, modified or qualified to be regulated for tolerance or to be aggressive to the antigen, using up of down regulation of polypeptide or polynucleotide encoded by or derived from axotrophin. The ex vivo derived differentiated clones may be propagated and may be used to treat the recipient, especially the original donor, to regulate the immune response. For example; a recipient may be rendered specifically tolerant to a foreign organ allograft prior to receiving the organ graft itself.

The modulation or inducing of an immune response in the methods of the present invention may be provided by polypeptide or polynucleotide encoded by or derived from axotrophin, analogs including fragments and fusion proteins, antibodies and other binding proteins and chemical compounds that directly inhibit or activate the polypeptides of axotrophin activity in the immune response.

Polynucleotide molecules and vectors according to the present invention may be provided in isolated and/or purified form, for example in substantially pure or homogeneous form. The term “isolate” may be used to reflect all these possibilities.

A peptide, polypeptide, antibody, polynucleotide or other molecule or agent for use in accordance with the present invention may be formulated into a composition, and is useful in pharmaceutical contexts.

The present invention also relates to a composition containing isolated axotrophin or a polypeptide or polynucleotide encoded by or derived from axotrophin and a pharmaceutically acceptable diluent, carrier or excipient which is suitably non-toxic and should not interfere with the efficacy of the active ingredient. The precise nature of the carrier or other material may depend on the route of administration, for example oral, intravenous, cutaneous or subcutaneous, nasal, intramuscular, intraperitoneal routes.

The diluent, carrier or excipient may be in the form of a gel, an oil or a liposome and, independently, preferably comprises a hydrophilic material, for example water. The precise nature of the carrier or other material may depend on the route of administration, for example oral, intravenous, cutaneous or subcutaneous, nasal, intramuscular, intraperitoneal routes.

Compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable ails, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, the active ingredient will suitably be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

The composition may be administered alone or in combination with other treatments, either simultaneously or sequentially dependent upon the condition to be treated and the availability of alternative or additional treatments.

In the present invention, a composition may be administered to an individual, particularly human or other primate. Administration may be to a human or another mammal, for example rodent such as mouse, rat or hamster, guinea pig, rabbit, sheep, goat, pig, horse, cow, donkey, dog or cat. Delivery to a non-human mammal need not be for a therapeutic purpose, but may be for use in an experimental context, for instance in investigation of mechanisms of immune responses to an antigen of interest, for example protection against cancers, pathogens and so on.

This invention is particularly useful for screening chemical compounds by using axotrophin or polynucleotides or polypeptides encoded by or derived from axotrophin or binding fragments thereof in drug screening techniques.

The invention provides a method of screening chemical compounds comprising contacting a test sample containing one or more chemical compounds to be screened with a binder selected from axotrophin, a polynucleotide or polypeptide encoded by or derived from axotrophin and fragment of such polynucleotide or polypeptide and determining whether the chemical compound has bound to the binder.

The binder may be in any suitable form including a vector, cell or composition and utilized in known ways of screening for chemical compounds.

The polypeptides polynucleotides or fragments employed in such a test may either be free in solution, affixed to a solid support, borne on a cell surface or located intracellularly. One method of drug screening utilizes eukaryotic or prokaryotic host cells which are stably transformed with recombinant polynucleotides expressing the axotrophin polypeptide or a fragment thereof. Chemical compounds may be screened against such transformed cells in competitive binding assays. Such cells, either in viable or fixed form, may be used for binding assays in a known manner.

Isolated proteins and polynucleotides of axotrophin may be used to obtain and identify agents which bind to a polypeptide encoded by or derived from an open reading frame (“ORF”) corresponding to axotrophin or bind to a specific domain of the polypeptide encoded by or derived from axotrophin.

The invention provides a screening method for identifying an agent which binds to axotrophin or a polypeptide or polynucleotide encoded by or derived from axotrophin comprising:
(a) contacting an agent with axotrophin or a or polynucleotide polypeptide encoded by or derived from axotrophin;
(b) determining whether the agent binds to the said polynucleotide or polypeptide; and
(c) detecting the formation of a complex, formed between the agent and the said polynucleotide or polypeptide such that if a complex is formed, the agent is detected.

In a preferred screening method the compound is contacted with a polypeptide or polynucleotide of axotrophin in a cell for a time sufficient to form a polypeptide complex of the compound with the polypeptide or polynucleotide, wherein the complex drives expression of a receptor gene sequence in the cell, and detecting the complex by detecting reporter gene sequence expression.

The invention also provides a kit comprising axotrophin or a polynucleotide probes and/or monoclonal antibodies, and optionally quantitative standards, for carrying out methods of the invention.

The present invention further provides a diagnostic method to identify the presence or expression of axotrophin or a polypeptide or polynucleotide encoding axotrophin in a test sample, using a polynucleotide probe or antibodies to axotrophin, optionally conjugated or otherwise associated with a suitable label.

The invention provides a diagnostic method for detecting axotrophin or a polynucleotide or polypeptide encoded by or derived from axotrophin comprising:

(a) contacting a sample to be tested for the presence of a polynucleotide or polypeptide encoded by or derived from axotrophin with a compound that binds to a polynucleotide or polypeptide encoded by or derived from axotrophin;

(b) determining whether the compound binds to a component of the sample; and (c) detecting the formation of a complex, formed between the agent and the protein or polynucleotide and such that if a complex is formed, the polypeptide or polynucleotide is detected.

Preferably the diagnostic method comprises contacting a sample under stringent hybridization conditions with polynucleotide primers that anneal to a polynucleotide of axotrophin and amplifying annealed polynucleotides, so that if a polynucleotide is amplified, a polynucleotide of axotrophin is detected in the sample.

In a preferred embodiment, the diagnostic method for assessing the immune response of an individual comprises obtaining a test sample from the individual, for example blood, incubating the test sample with one or more of the antibodies or one or more of a polynucleotide probes for axotrophin or a polynucleotide or polypeptide encoded or derived from axotrophin and assaying for binding of the polynucleotide probes or antibodies to components within the test sample.

Assays according to embodiments of the present invention may employ ELISA, Western blot, immunohistochemistry, identification of the effects of drugs on the immune response in terms of induced bias towards regulatory tolerance, anergy or deletion, versus rejection and any other suitable technique available in the art.

Tests may be carried out on preparations containing cDNA and/or mRNA. RNA is more difficult to manipulate than DNA because of the wide-spread occurrence of RN′ases, which is one reason why CDNA analysis may be performed.

However, since it will not generally be time or labour-efficient to sequence all polynucleotide in a test sample or even the whole gene of interest, a specific amplification reaction such as PCR using one or more pairs of primers may be employed to amplify the region of interest in the polynucleotide if present in the sample. This may be done quantitatively, allowing for determination of the amount of axotrophin or a polypeptide or polynucleotide encoded by or derived from axotrophin in the test sample.

Polynucleotide may be screened using a specific probe Such a probe corresponds in sequence to a region of the relevant gene, or its complement Under suitably stringent conditions, specific hybridisation of such a probe to test polynucleotide is indicative of the presence of the polynucleotide molecule of interest, and again this may be quantitated to provide an indication of the amount of such polynucleotide molecule in the test sample.

Specific oligonucleotide primers may similarly be used in PCR to specifically amplify particular sequences if present in a test sample.

A method may include hybridisation of one or more (for example two) probes or primers to target polynucleotide. Where the polynucleotide is double-stranded DNA (e.g. cDNA), hybridisation will generally be preceded by denaturation to produce single-stranded DNA, The hybridisation may be as part of a PCR procedure, or as part of a probing procedure not involving PCR. A screening procedure, chosen from the many available to those skilled in the art, is used to identify successful hybridisation events and may allow for quantitation of the amount of polynucleotide present in the original sample.

Binding of a probe to target polynucleotide (for example DNA) may be measured using any of a variety of techniques at the disposal of those skilled in the art. For instance, probes may be radioactively, fluorescently or enzymatically labeled. Probing may employ a standard blotting technique.

A test sample of polynucleotide may be provided for example by extracting polynucleotide from cells such as spleen cells or biological tissues or fluids, urine, saliva, faeces, a buccal swab, biopsy or blood.

A test sample may be tested for the presence of a binding partner for a specific binding member such as an antibody molecule (or mixture of antibodies), specific for the polypeptide or polypeptide of interest. The sample may be tested by being contacted with a specific binding member such as an antibody molecule under appropriate conditions for specific binding, before binding is determined, for instance using a reporter system as discussed. Where a panel of antibodies is used, different reporting labels may be employed for each antibody so that binding of each can be determined.

A specific binding member such as an antibody molecule may be used to isolate and/or purify its binding partner polypeptide from a test sample, to allow for sequence and/or biochemical analysis of the polypeptide to determine whether it has the sequence and/or properties of axotrophin or a polypeptide or polynucleotide encoded by or derived from axotrophin. Amino acid sequencing is routine in the art using automated sequencing machines.

A test sample containing one or more polypeptides may be provided for example as a crude or partially purified cell or cell lysate preparation, for example using tissues or cells, such as from the spleen or a bodily fluid, preferably blood.

Other tests may involve the use of blood or spleen cells taken from a test animal, individual, subject or patient, and ex vivo challenge of the cells with antigen to determine the presence or absence of an aggressive or tolerant response to the antigen.

Suitable probes may, for example, be used to determine whether specific mRNA molecules are present in a cell or tissue or to isolate similar polynucleotide sequences from chromosomal DNA, for example as described by Walsh et al. (Walsh, P. S. et al., 1992, PCR Methods Appl 1:241-250). They may be labeled by nick translation, Kienow fill-in reaction, PCR, or other methods known in the art. Suitable probes, their preparation and/or labeling are elaborated in Sambrook, J. et al., 1989, Molecular Cloning: A Laboratory Manual, Cold Spring Harbor Laboratory, NY; or Ausubel, F. M. et al., 1989, Current Protocols in Molecular Biology, John Wiley & Sons, New York N.Y. All documents mentioned anywhere in this specification are incorporated by reference. The invention is illustrated by the following non-limiting Examples and accompanying Figures.

### EXAMPLE 1

Transplantation Tolerance: Gene Expression Profiles Comparing allo-tolerance Versus allo-jection

In mice, infectious regulatory tolerance is inducible by CD4/CD8 blockade in recipients of vascularised heart grafts. Once established, this transplantation tolerance is robust and isolated “tolerant” spleen cells show powerful immune regulatory properties, being able to impose donor-specific allo-tolerance upon fully immune competent naive recipients. Using ^{BALB/c-tolerant}CBA (H-2^{k}) mice, we analysed spleen cell responses to donor (BALB/c [H-2^{d}]) antigen at a series of time points and in comparison with an identical ex vivo series of ^{BALB/C-rejected}CBA spleen cells. The key feature of rejection was rapid Interferon gamma release. In contrast, Interferon gamma in tolerance was low and less than that released in response to third party antigen (C57Bl10 [H-2^{b}]). Positive markers of primed tolerance were high expression of STAT3 and c-kit, and release of LIF. Here we present a compound comparison of four gene arrays (tolerance versus rejection, at 48h, and at 123h) where a relatively small number of differentially expressed genes occurred. In rejection, there was a strong progressive amplification of Interferon gamma and granzyme B mRNAs. In tolerance, both Emk and axotrophin were upregulated at 123h. Mice lacking Emk develop auto-immune disease (Hurov et al, Mol Cell Biol, 2001). Mice lacking axotrophin show abnormal axonal migration during development. Taken together, our results suggest a link between developmental regulation and immune regulation, and highlight a possible role for axotrophin in regulatory tolerance.

Materials and Methods

Generation of ^{BALB/c-primed} CBA Mice.

CBA mice (H2^{k}) of 10-12 weeks of age received a fully mismatched, vascularised BALB/c (H2^{d}) heart graft to the neck, using the technique described by Chen, [Chen Z. K., Cobbold, S. P., Waldmann, H. & Metcalfe, S. M. Amplification of natural regulatory immune mechanisms for transplantation tolerance. Transplantation 62, 1200-1206 (1996)]. Tolerance was generated by a 21 day course of alternate day therapy using blocking mAbs to CD4 and CO8 as previously described [Chen, Z. K,, Cobbold, S. P., Waldmann, H, & Metcalfe, S. M. Amplification of natural regulatory immune mechanisms for transplantation tolerance. Transplantation 62, 1200-1206 (1996)]. ^{BALB/c-tolerant}CBA spleen cells from tolerant recipients were isolated at least 100d after grafting for ex vivo analyses. For comparison, untreated CBA mice, 10-12 weeks of age, were grafted either with BALB/c tail skin which rejected by day 10, or with a BALB/c heart which rejected on day 7. The ^{BALB/c-rejected}CBA spleen cells were collected at 14d for ex vivo analyses. All procedures were carried out according to Home Office licence under the Animals (Scientific Procedures) Act 1986, UK.

Ex vivo Cultures

Culture conditions have been described in detail elsewhere [Metcalfe, S. M. & Moffatt-Bruce, S. D. An ex vivo model of tolerance versus rejection: Comparison of STAT1, STAT4, STAT5 and STAT6. Clin. Chem. and Lab. Med. 38, 1195-1199 (2000)]. Briefly, responder spleen cells were obtained from either ^{BALB/c-tolerant}CBA, or ^{BALB/c-rejected}CBA, mice, and the tolerant and rejected cell populations were stimulated ex vivo by irradiated BALB/c spleen cells (donor antigen), using 4×10⁷ responders to 6×10⁷ stimulators in a total of 10 ml growth medium supplemented with 10% FCS. After 48h, one flask each of tolerant and rejected spleen cells were removed for total RNA preparation. A second pair of flasks (one tolerant, one rejected) were boosted with a further 7×10⁷ stimulator spleen cells at 120 h, and then harvested at 123 h. At harvest, cells were collected onto ice, with any adherent cells being included following brief treatment with 0.25% trypsin. After resuspending the cells to homogeneity, a 1.5 ml aliquot was removed for RNA extraction. After washing in ice cold 0.1% BSA/PBS, the cells were collected into sterile 15 ml Falcon centrifuge tubes and pelleted at 1600rcf for 5 min at +4° C. Supernatant was discarded and the tube wiped clear of supernatant residue prior to resuspending the cells in pre-cooled Trizol reagent, vortexed, and then immediately stored at −80° C. One ml Trizol was used per 6×10⁻⁶ cells.

RNA Isolation.

Samples were brought to room temperature and kept for 10 minutes before addition of 1 ml chloroform and vortexing to an emulsion. After 15 min the samples were centrifuged at 1600rcf for 10 min at 40C. The upper phase was transferred to RNA-ase-free Eppendorff tubes in 400 μl aliquots and an equal volume of isopropanol added. After gentle mixing and standing for 15 min, the samples were centrifuged at 13,000 g at 40C for 10 min. The supernatant was removed and discarded. The RNA pellet was washed in 350 μl of 75 ethanol and sedimented at 7500 g for 5 min at 40C. The supernatant was aspirated and the pellet air dried for 20 min. The aliquoted RNA pellets were collected together for each sample by dissolving and serial transfer of 50 μl DH₂O; a second 50 μl was used to serially collect washings from each tube, giving a final total sample volume of 100 μl in DH₂O. This was stored at −80° C. until transfer to the MRC HGRC at Hinxton Hall for customer service preparation of cRNA and array using Affymetrix U74 chips by standard methodologies.

Gene Array.

Analyses of the combined arrays was prepared using dChip software [Wong, C.U.W.H., PNAS USA, 98, 31, 2001].

### RESULTS

Combined 48 h and 123 h arrays of the matched tolerant and rejected samples pairs gave 129 genes showing differential expression. To identify those genes that showed a biased expression in either tolerance, or in rejection, the results were ranked in three ways: those genes showing a positive shift from 48 h to 123 h (Table 1); those genes with high expression at 123 h (Table 2); and those genes (tolerant) that showed a positive shift, whilst the rejection counterpart showed a negative shift from 48 h to 123 h (Table 3).

Of the genes that increased in expression from 48 h to 123 h, 10 were in the tolerant cultures with increases ranging from 1.71 fold to 4.00 fold. Expression of the same genes in the rejection response showed either no increase in expression or a decrease in expression (Table 1(a)). Of particular note was axotrophin, a newly discovered stem cell gene; cyclin B2, associated with the cell cycle and cellular migration; histone H2A-X that may play a role in chromatin remodeling; and ELKL motif kinase, also known as Erk, required to regulate the immune response and protect against auto-immunity. Table 1 (b) shows the 5 genes that increased in expression in rejection. Again this increase was specific to rejection, with the exception of granzyme B with a twofold increase in both tolerance and rejection; however, the actual levels of granzyme B mRNA were six times greater in rejection than in tolerance. The 12-fold increase in Interferon gamma mRNA in rejection was in accord with our previous findings of high Interferon gamma protein release in these cultures.

Of those genes that showed high expression at 123 h, within the context of the four arrays, 15 were in the tolerant set (Table 2(a)) and included axotrophin. In rejection, 13 genes are ranked in order of expression level in Table 2(b) with granzyme B and Interferon gamma being the highest. This analytical approach therefore showed correlation with phenotype with respect to granzyme B and Interferon gamma, and again placed axotrophin as being associated with tolerance, although the actual expression level was not great A further analysis was made, identifying those genes that showed increased expression in tolerance whilst showing a decreased expression in rejection (Table 3). This revealed Histone H2A-X, involved in chromatin structure and remodeling; ELKL motif kinase; splicing factor 3b subunit 1 (SF3b-155), acting as part of the mRNA splicing complex and probably involved in exon removal; and cyclin B2, a regulator of the cell cycle and also involved in cellular migration when complexed with cdc2.

TABLE 1a and 1b
Genes showing increased expression {48 h versus 123 h)

| | | | |
|---|---|---|---|
| | | Tolerance: | Rejection: |
| | Accession | Fold | Fold |
| Gene | Number | increase | increase |

TOLERANCE

| | | | |
|---|---|---|---|
| Dual specificity phosphatase 1 | X61940 | 4.00 | 0.99 |
| BCL2-like 11 | AA796690 | 3.11 | 1.15 |
| Axotrophin* | AW212859 | 2.9 | 1.00 |
| H2A histone family, member X | M33988 | 2.22 | 0.46 |
| Interferon stimulated protein | AW122677 | 2.21 | 0.95 |
| (20 kDa) | | | |
| Chemokine (C-C) receptor 6 | AJ222714 | 2.02 | 0.95 |
| Cyclin B2 | X66032 | 2.01 | 0.59 |
| Paneth cell enhanced expression | U37351 | 2.0 | 0.98 |
| Splicing factor 3b, sub-unit 1, | A1844532 | 1.93 | 0.59 |
| 155 kDa | | | |
| ELKL motif kinase** | X70764 | 1.71 | 0.63 |

REJECTION

| | | | |
|---|---|---|---|
| Interferon gamma | K00083 | 0.69 | 11.98 |
| Glutaryl CoA dehydrogenase | U18992 | 1.20 | 5.10 |
| CD3 antigen, gamma polypeptide | M18228 | 1.23 | 3.22 |
| Interleukin 1 receptor antagonist | L32838 | 1.00 | 2.57 |
| Granzyme B | M12302 | 2.07 | 2.52 |

TABLE 2a and 2b
Genes showing high expression at 123 h
within the context of the four arrays

| | | |
|---|---|---|
| | | Expression |
| | Accession | level @ |
| Gene | Number | 123 h |

TOLERANCE

| | | |
|---|---|---|
| □-2 microglobulin | X01838 | 9047 |
| Ring Finger protein 10 | AB026621 | 4127 |
| CD53 antigen | X97227 | 3927 |
| Guanylate nuceotide binding protein 1 | M55544 | 1005 |
| Spermidine spermineN1 acyl transferase | L10244 | 1002 |
| Glycoprotein 49A | M65027 | 975 |
| Chemokine (C-C) receptor 6 | AJ222714 | 972) |
| BCL2-like 11 | AA796690 | 752 |
| Paneth cell enhanced expression | U37351 | 753 |
| EST | AW047461 | 744 |
| Chemokine (C-C motif) ligand 9 | C-U49513 | 593 |
| EST | A1060627 | 562 |
| Dual specificity phosphatase 1 | X61940 | 536 |
| Expressed Sequence AU021774 | A1854141 | 438 |
| Axotrophin* | AW212859 | 416 |

REJECTION

| | | |
|---|---|---|
| Granzyme B | M12302 | 6766 |
| Interferon gamma | K00083 | 3103) |
| Metallothionein 2 | KO2236 | 1952 |
| Lectin, galactose binding, soluble 1 | X15986 | 1887 |
| RNA binding motif protein 3 | AB016424 | 1725 |
| Acidic nuclear Phosphoprotein 32 family, | A1842771 | 1665 |
| member B | | |
| Glutaryl-Coenzyme A dehydrogenase | U18992 | 1350 |
| STAT3 | U08378 | 1026 |
| STAT5A | AJ237939 | 988 |
| Calcylcin | X66449 | 856 |
| CD3 antigen pyrophosphate | M18228 | 517 |
| IL1 receptor antagonist | L38838 | 511 |
| Exp Sequence AU044919 | X67210 | 356 |

TABLE 3
Genes showing increases in expression in tolerance
and decreased expression in rejection

| | Accession | |
|---|---|---|
| Gene | Number | Gene description |
| H2A histone family, | M33988 | Chromatin remodeling (Bassing; |
| member X | | Bruno) |
| ELKL motif kinase** | X70764 | Immune regulation ((Hurov) |
| Splicing factor 3b, | A1844532 | RNA splicing, intron removal (Horie) |
| subunit 1, 155 kDa | | |
| Cyclin B2 | X66032 | Cell cycle; cell migration (Manes) |

### EXAMPLE 2

The Stem Cell Gene axot is Associated with Regulation of LIF and Mitogenic Activation of T Lymphocytes.

Control of “stemness”¹ for self-renewal of stem cells, versus their differentiation during organogenesis, is fundamental to the new field of regenerative medicine, Leukaemia inhibitory factor (LIF) is critical to this control, acting as a suppressor of stem cell differentiation ^{2,3}. The finding that both LIF and axot, a novel stem cell gene^{1,4}, are linked also to immune tolerance suggests a relationship between sternness and immunity. To explore this relationship we have asked if immune cells from axot^{-/-} mice differ from those of axot^{+/+} littermates. We discovered (i) that presence of axotrophin is involved in damping down proliferation of T, but not B, lymphocytes; (ii) that lack of axotrophin leads to excessive release of T cell cytokines: and (iii) an axot gene-dose dependent suppression of LIF. This is the first evidence that fate determination mediated by LIF maybe linked to axotrophin, and demonstrates commonalities between sternness and immune tolerance that may favour acceptance of implanted stem cell allo-grafts for therapeutic tissue regeneration.

Fate determination in stem cells is a critical feature in development, providing q balance between pluripotent self-renewal versus differentiated function within the whole organism. In regenerative medicine, understanding the molecular basis of fate determination of stem cells is important if they are to be used successfully in the treatment of disease. Fate determination pathways also play a key role in the immune system, where reactivity is finely tuned to ensure protective tolerance towards self tissues whilst simultaneously being capable of aggressive attack towards foreign pathogens. Although the regulatory tolerance pathway is little understood, the recent demonstration that a single gene, foxp3, is able to orchestrate the differentiation of naive CD4+T cells into regulatory T cells (Treg)^{5,6,7} implies the existence of “master” switches for fate determination in immunity. We have recently discovered features of immune tolerance that are common to regulation of stem cell fate, raising two important questions: do “stemness” signals play a role in autoimmunity by suppressing terminal differentiation of immune effector cells? Do allogeneic stem cells bias the alto-immune response towards allo-tolerance, by signaling for “stemness”, so favouring successful therapeutic engraftment? This paper describes how we discovered that axotrophin, expressed in embryonic, neuronal, and haematopoletic stem cells¹, is not only involved in regulation of T lymphocyte reactivity, but also in regulation of LIF, thereby providing a novel concept of immunoregulation.

The molecular events associated with immune tolerance, versus immune aggression, have been compared in previous studies using an ex vivo model⁸. This is derived from mice where fully mismatched heart grafts, normally rejected by day 7, become accepted indefinitely after short term blockade of CD4 and CD8 (ref. 9). Once established, this transplantation tolerance is self-perpetuating and isolated “tolerant” spleen cells show powerful immune regulatory properties, being able to impose donor-specific alto-tolerance when infused into fully immune competent naive recipients. We characterised the ex vivo responses of the tolerant spleen cells, versus spleen cells from mice that had been primed to reject the same donor-type and the key features of rejection were rapid interferon gamma release and strongly amplified expression of genes encoding Interferon gamma and granzyme B. In marked contrast, tolerance showed features in common with sternness, these being the release of LIF and increases in c-kit (the receptor for stem cell factor (SCF)) and in STAT3 (signal transducer and activator of transcription 3, responsive to both SCF and LIF activity). We found that the relationship between LIF and tolerance was also evident in cloned Treg, showing high levels of LIF release in contrast to Th1 and Th2 clones. At the gene level, tolerance was associated with strong induction of a newly discovered stem cell gene, axot (Genbank accession number AF155739). To test of our hypothesis that sternness and tolerance are linked, we have asked if axotrophin influences immune responsiveness.

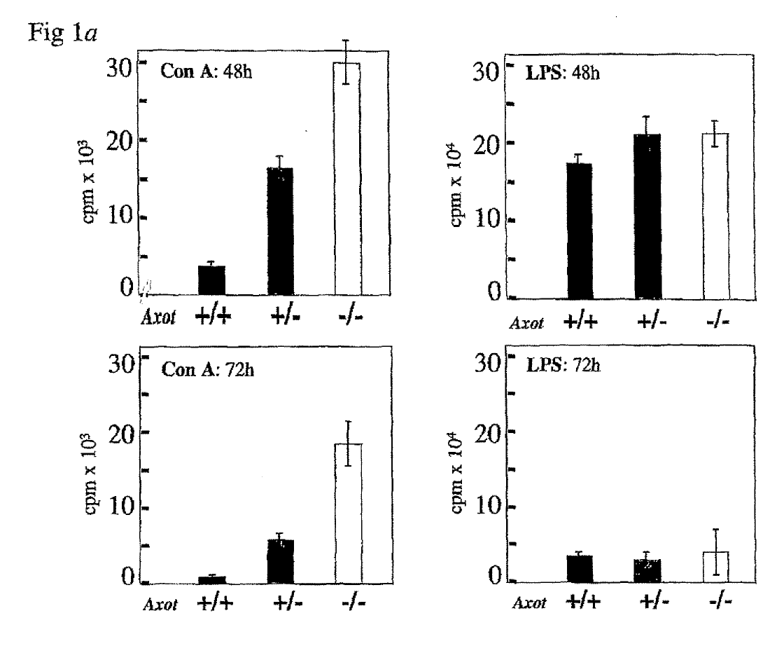

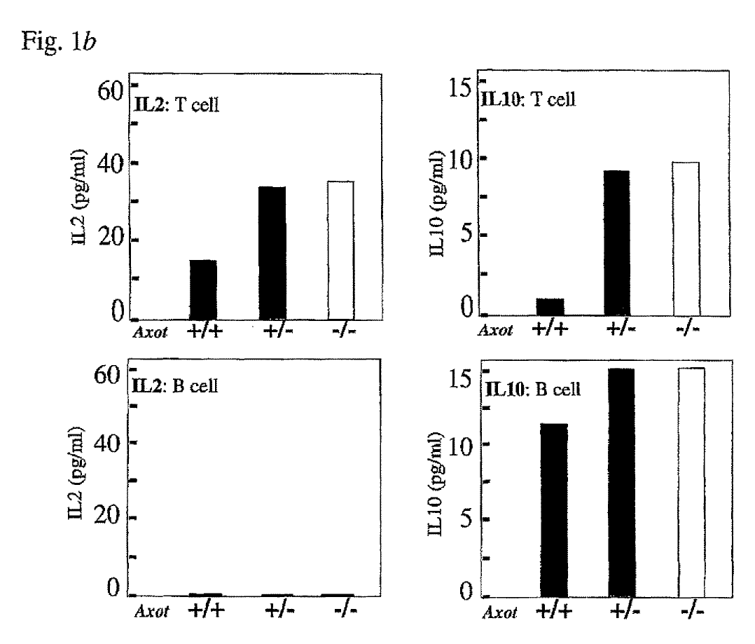

We first looked at lymphocyte responsiveness to mitogen. Axot null (axot^{-/-} mice were compared to littermates that expressed either one axot allele (heterozygous, axot^{+/-}) or both alleles (wild-type; axot^{+/+}). Whole cell populations were freshly isolated from the spleen and we measured mitogenic activation using either concanavalin A (conA) as a T cell mitogen, or lipopolysaccharde (LPS) as a B cell mitogen. We also looked for any kinetic effects on responsiveness by comparing DNA synthesis at 48 h and at 72 h. Since activated lymphocytes show a synchronised entry into the cell cycle, with S phase peaking at 48 h (ref. 10), we reasoned that a consistent reduction in DNA synthesis in the axot null cells, compared to the axot^{+/+} cells, would indicate a loss of mitogenic responsiveness due to lack of axotrophin. However, the level of T cell proliferation showed a marked increase in the axot null cells when compared to wild-type cells. This was not caused by altered kinetics since the axot-related differentials were similar at both 48 h and 72 h (FIG. 1a, FIG. 1b). Therefore axotrophin appeared to be repressing the proliferative response of T cells. Moreover, since the heterozygous axot^{+/-} T cells showed intermediate hyper-proliferation, the repression appeared sensitive to axot gene dose. In marked contrast to the T cells, B lymphocyte proliferation was not significantly altered by axotrophin (FIG. 1c, FIG. 1d). We concluded that axotrophin plays a role in damping down T, but not B, lymphocyte proliferation following mitogenic stimulation. No spontaneous mitogenesis occurred in cultures of axot^{+/+}, axot^{+/-} or axot^{-/-} spleen cells over a 7 d period.

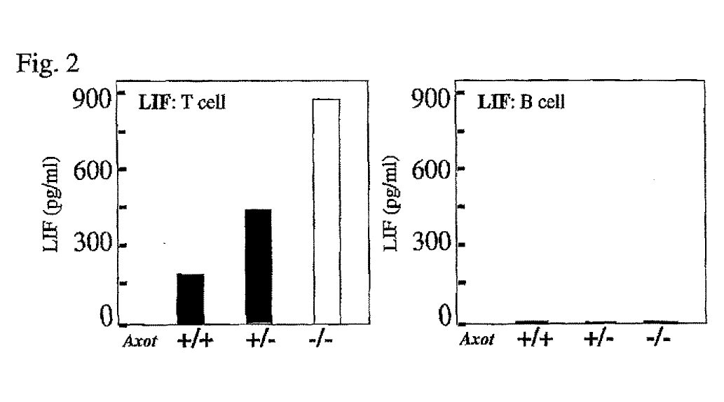

As a further test of functionality in the axot null spleen cells, we measured cytokine release in response to mitogen. Lack of axotrophin was associated with a twofold increase in interleukin 2 (IL2) following conA treatment, in both axot null and axot heterozygous cell cultures (FIG. 2a). This IL2 equivalence revealed that IL2 was not a limiting factor for T cell proliferation, where there had been a four-fold difference. Splenic B cells did not release IL2 (FIG. 2b) whilst both T and B cells released IL10 in response to their respective mitogens. Again, only the conA-treated cultures were affected by a lack of axotrophin, with a ten-fold increase in IL10 in both axot^{+/-} and axot^{-/-} cell cultures (FIG. 2c, FIG. 2d). These findings show that partial or total reduction of axotrophin results in a general increment in both IL2 and IL10 from activated T cells, but has no effect on IL10 release from activated B cells. Interferon gamma and IL4 were also measured and showed a similar axot-related increment to that found for IL2 in the conA-treated cultures, as detailed in the legend to FIG. 1. LPS-treated cultures were negative for Interferon gamma and IL4.

Unexpectedly, we found that release of LIF in response to conA was strongly inhibited by axotrophin and that this inhibition was gene-dose dependent (FIG. 2). There was no LIF in the LPS-treated cultures irrespective of axot genotype. Based on the relationship between LIF concentration versus axot gene dose, we have hypothesised that gene dose correlates with expression levels of axotrophin. Both LIF release and T cell proliferation would thus appear to be critically influenced by axotrophin and our results would be in accord with inter-dependent links between the three.

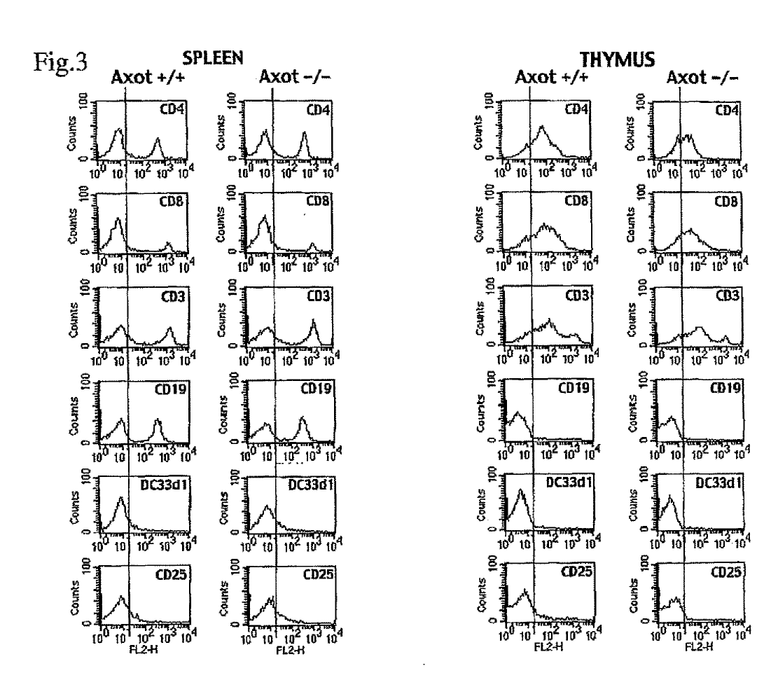

By analysis of phenotype and of histological structure, we looked for effects of axotrophin on the phenotypic composition of lymphoid organs. Cell sub-populations were identified by FACS analysis as follows: cells expressing the T cell markers CD3, CD4 and CD8; the B cell marker CD19:; the activation marker of T cells and of regulatory tolerant T cells, CD25; and markers of dendritic cells, CD205 and DC33D1. None of these markers showed differential expression between the axot^{+/+}, axot^{+/-}, and axot^{-/-} littermates (FIG. 3). Similarly, histological assessment of the spleen and thymus showed no significant differences between the three axot genotypes.

Fate determination is controlled by genetic programmes that are altered by changing the nature and frequency of cytokine interactions within the microenvironment, both for totipotent and pluripotent stem cells, and for the differentiation of precursor cells. LIF Is a key determinant of self-renewal of stem cells¹¹ in addition to being a neuropoietic cytokine¹². Having shown that axotrophin may act as a negative regulator of LIF, at least in activated T cells, we suggest that LIF expression is functionally coupled to axotrophin expression, with axotrophin playing a role in co-ordinating the positive and negative regulation of LIF release. This would place axotrophin as a potential regulator of fate determination via LIF. The molecular function of axotrophin has yet to be determined and how axotrophin might influence LIF release is unknown. Future work will include exploration of this relationship, looking for effects of axotrophin on LIF gene expression¹³, and on regulation of LIF-induced signaling through the LIF-R/gp130 complex^{14,15,16,17}.

As a working model we propose that LiF activity, regulated by axotrophin, is associated with immune tolerance. LIF may guide naive T cells towards a relatively undifferentiated, non-aggressive phenotype in response to presented antigen, where the circumstances of presentation initiate the tolerogenic LIF activity, either directly or indirectly (e.g. antigen presentation by immature or regulatory dendritic cells^{18,19} and associated vitamin D activity²⁰; or reduced T cell responsiveness due to altered function of CD4/CD8 (ref. 9) or CD28 (ref. 21)). Thereafter, epigenetic changes, including expression of foxp3 and ROG¹⁸, and induction of Id transcription factors²², would stabilise the tolerant phenotype for inheritable Treg activity. A link between stem cell biology and regulatory immune tolerance has direct relevance to therapeutic intervention of immune-related diseases and to immunosuppressive treatment of organ transplant recipients. The work also has major implications for use of stem cells for regenerative medicine, since the properties we have discovered may enhance successful outcome of implanted stem cells in patients.

In summary, we have discovered that axotrophin represses T lymphocyte proliferative responsiveness in adult mice and that axotrophin is able to act as a negative regulator of LIF, implying that axotrophin acts through LIF to regulate T cells.

### METHODS

Mice

Gene trap insertion was used to generate axot null BALB/C mice and littermates from heterozygous parents were genotypes by PCR analysis of genomic DNA to identify axot^{+/+}, axot^{+/-}, and axot^{-/-} pups as detailed previously. Spleen, thymus and lymph node were obtained from 5 m old littermates and kept on ice prior to cell preparation for the analyses described below. The lymph node tissue yielded very few cells and was discarded. Spleen and thymus from axot^{+/+}, axot^{+/-}, and axot^{-/-littermates} were also taken for histology. These were bisected and fixed in 70% ethanol. Fixed tissues were embedded in paraffin blocks and sectioned, then stained with haematoxylin and eosin using standard procedures.

Proliferation Assays

Splenocytes and thymocytes were teased out from each organ and collected in sterile growth medium [RPMI-1640 (Gibco™ Invitrogen Co.) supplemented with 10% FCS (Gibco™ Invitrogen Co.), 200 mM L-Glutamine, 100 U/mL Penicillin and 100 μg/mL Streptomycin (Sigma Chemical Co.)]. The cell suspensions were washed, resuspended in growth medium and counted using a haemocytometer.

The cells were seeded in 100 μl growth medium at 5×10⁵ nucleated cells per well in flat bottomed 96-well Nunclon™ tissue culture plates and incubated at 37° C., 5% CO₂ for 48 h or 72 h. LPS, (Sigma Chemical Co.) at 50 μg/mL and conA (ICN Biochemicals, USA) at 10 μg/mL, were added as mitogens at time zero. All experiments were performed in triplicate. Immediately prior to harvest, supernatants were collected for ELISA analysts and the cells were incubated for 2 hrs in pre-wan-ned GM containing methyl-[³H] Thymidine (TRK686, specific activity 80Ci/mmol, Amersham Biosciences) at a final concentration of 1 μCi/mL. Cells were harvested using a Filtermate196, Packard harvester and counted using a Packard TopCount.NXT™ microplate scintillation and luminescence counter.

To determine the effect of LIF on Con A stimulation, BALB/c axot^{+/+} splenic and thymic cells were incubated in the presence of Con A (2 μg/mL or 10 μg/mL) together with 500 pg/mL or 1000 pg/mL rmLIF (Santa Cruz Biotechnology, SC-4378). Mitogensis was measured as described above. Controls included GM only, conA only, and LIF only, at the respective concentrations.

Elisa

ELISA's were performed on the 48 h culture supernatants, in 96-well Falcon® plates using the DuoSet® ELISAS for Interferon gamma (DY4855, IL2 (DY402), IL4 (DY404), IL10 (DY417) and Quantikine®M Immunoassay for LIF (MLF00), from R&D Systems. The standard curves were established by processing the optical density data using Microsoft Excel software and cytokine concentrations were determined using the standard curves.

Flow Cytometry

The splenic and thymic cell suspensions were RBC depleted and washed in FACS staining solution (0.2% BSA and 0.1% sodium azide in 1×PBS) prior to being mixed with the various monoclonal antibodies detailed below, these being either directly or indirectly conjugated with Phycoerythrin (PE) or Fluorescein isothiocyanate (FITC), PE-rat anti-mouse CD19 (557399), PE-hamster anti-mouse TCRα chain (553172) and rat anti-mouse dendritic cell clone 33D1 (551776) were from Pharmingen. Rat anti-mouse CD205-FITC (MCA949F), mouse anti-rat IgG2a heavy chain-FITC (MCA278F) and mouse anti-rat IgG2b chain-FITC were from Serotec Ltd. while rabbit anti-mouse CD25 (IL2Rα) and goat anti-rabbit IgG (H&L)-PE (4050-89) were from Santa Cruz Biotechnology and Southern Biotechnology Associates respectively. Anti CD4 (YTS177.9.6) and anti CD8 (YTS 105.18.10) were a gift from Professor Stephen Cobbold, University of Oxford. Analyses were performed on a Becton Dickinson FACSCalibur instrument equipped with CellQuest software.

### REFERENCES

1. Ramalho-Santos, M., Yoon, S., Matsuzaki, Y., Mulligan, R. C. & Melton, D. A. “Stemness”: transcriptional profiling of embryonic and adult stem cells. Science 298, 597-600 (2002).

2. Murray, P. & Edgar, D. The regulation of embryonic stem cell differentiation by leukaemia inhibitory factor (LIF). Differentiation 68, 227- 234 (2001).

3. Viswanathan, S. et al. Supplementation-dependent differences in the rates of embryonic stem cell self-renewal, differentiation, and apoptosis. Biotechnol. Bioeng. 84, 505-517 (2003).

4. Baker, R. K. et al In vitro preselection of gene-trapped embryonic stem cells for characterising novel developmentally regulated genes in the mouse. Dev Biol. 185, 201-214 (1997).

5. Hori, S. et a/, Control of regulatory T cell development by the transcription factor Foxp3. Science 299,1057-1061 (2003).

6. Fontenot, J. D., Gavin, M. A., & Rudensky, A. Y. Foxp3 programs the development and function of CD4+CD25+ regulatory T cells. Nat. Immunol. 4, 330-336 (2003).

7. Khaltri R., Cox, T., Yasayko, S. A. & Ramsdell, F. An essential role for Scurfin in CD4+CD25+ T regulatory cells. Nat. Immunol. 4, 337-342 (2003).

8. Metcalfe, S. M. & Moffatt-Bruce, S. D. An ex vivo model of tolerance versus rejection: Comparison of STAT1, STAT4, STAT5 and STAT6. Clin. Chem. and Lab. Med. 38, 1195-1199 (2000)

9. Chen, Z. K., Cobbold, S. P., Waldmann, H. & Metcalfe, S. M. Amplification of natural regulatory immune mechanisms for transplantation tolerance. Transplantation 62, 1200-1206 (1996).

10. Milner, S. M. Activation of mouse spleen cells by a single short pulse of mitogen. Nature 268, 441-442 (1977).

11. Zandstra, P. W. Le ,H.V,, Daley, G. Q., Griffith, L. G. & Lauffenburge, D. A. Leukemia inhibitory factor (LIF) concentration modulates embryonic stem cell self-renewal and differentiation independently of proliferation. Biotechnol. Bioeng. 69, 607-617 (2000).

12 Patterson, P. H. Leukemia inhibitory factor, a cytokine at the interface between neurobiology and immunology. Proc. Natl. Acad. Sci. 911 7833-7835 (199).

13. Bamberger, A. M., et al. Regulation of the human leukemia inhibitory factor gene by ETS transcription factors. Neuroimmunomodulation 11, 10-19. (2004)

14. Moon, C. et a/. Leukemia inhibitory factor inhibits neuronal terminal differentiation through STAT3 activation. Proc. Natl. Acad. Sci. 991 9015-9020 (2002).

15. Cheng, J. G., Chen, J. R., Hernandez, L., Alvord, W. G. & Stewart, C. L. Dual control of LIF expression and LIF receptor function regulate Stat3 activation at the onset of uterine receptivity and embryo implantation.
Proc. Natl. Acad. Sci. 98, 8680-8685 (2001).

16. Takahashi, Y. et a/. SOCS3: an essential regulator of LIF receptor signaling in trophoblast giant cell differentiation. EMBO J 22, 372-384 (2003).

17. Bartoe, J. L. & Nathanson, N. M. Independent roles of SOCS-3 and SHP-2 in the regulation of neuronal gene expression by leukemia inhibitory factor. Brain Res Mol Brain Res. 107,109-119 (2002).

18. Cobbold, S. P. et al. Regulatory T cells and dendritic cells in transplantation tolerance: molecular markers and mechanisms. Immuno./Rev. 196,109-124(2003).

19. Jeudes, A. E. & Von Herrath, M. G. Using regulatory APCs to induce/maintain tolerance. Ann. N.Y. Acad. Sci. 1005, 128-137 (2003).

20. Adorini L. Tolerogenic dendritic cells induced by vitamin D receptor ligands enhance regulatory T cells inhibiting autoimmune diabetes, Ann. N. Y. Acad. Sci. 987, 258-261 (2003).

21. Tang, Q. et al. CD28 controls peripheral homeostasis of CD4+CD25+ regulatory T cells. J. Immunol. 171, 3348- 3352 (2003).

22. Ying, Q. L.₇ Nichols, J., Chambers, I. & Smith, A. BMP induction of Id proteins suppresses differentiation and

Figure Legends

FIG. 1.

DNA Synthesis and Cytokine Release by Splenocytes from axot^{+/+}, axot^{+/-}, and axot^{-/-} Littermates

(a) H³-thymidine labeling of spleen cells stimulated for 48 h (upper panels) or 72 h (lower panels) with conA (left-hand panels) or LPS (right-hand panels) DNA synthesis and standard deviation are shown after subtraction of the respective background controls for each genotype. Background controls were all less than 300 cpm. (b) levels of IL2 and IL10 in supernatants at 48 h after stimulation with either conA (upper panels) or LPS (lower panels). Interferon gamma and IL4 were also measured: Interferon gamma was present in the conA culture supernatants only, the concentrations being 538 pg/ml, 1410pg/ml, and 909 pg/ml respectively for axot^{+/+}, axot^{+/-}, and axot^{-/-} cultures. IL4 was also only found in the conA supernatants and was 121 pg/ml, 263 pg/ml, and 92 pg/ml respectively for axot^{+/+}, axot^{+/-}, and axot^{-/-} cultures. The regression analyses for goodness of fit of each ELISA were as follows, IL2, R²=0.946; IL4, R²=0.925, IL10, R²=0.939; and Interferon gammaR²=0.937.

FIG. 2.

Effect of axotrophin on LIF Release.

LIF release from spleen cells of axot^{+/+}, axot^{+/-}, and axot^{-/-} littermates after 48 h conA (left panel) or 48 h LPS (right panel) stimulation. The regression analyses for goodness of fit was R²=0.999.

FIG. 3.

Phenotypic Profile of Spleen and Thymus from axot^{+/+}, and axot^{-/-} Mouse Littermates.

Whole populations of spleen and thymic cells were prepared, stained and analysed as described in Materials and Methods. The FACs data is presented in histogram format with the cut-off for negative staining indicated by the vertical line through each data set of CD4, CD8, CD3, CD19, DC33d1, and CD25 staining. The mouse axot^{+/+} and axot^{-/-} genotypes are as indicated above each panel. Axot^{+/-} splenocytes and thymocytes were also analysed and gave the same results as those shown. CD205 staining was negative throughout.

1 . Use of axotrophin or a polypeptide or polynucleotide encoded by or derived from axotrophin to induce or to regulate, directly or indirectly, the immune response of an individual and/or ex vivo cell population to an antigen.

2. Use of axotrophin or a polypeptide or polynucleotide encoded by or derived from axotrophin in the manufacture of a medicament to induce or to regulate, directly or indirectly, the immune response of an individual and/or ex vivo cell population to an antigen.

3. Use according to claim 1 or claim 2 in which the immune response is in a vertebrate wherein the individual has a tissue or cell transplant.

4. Use in the manufacture of a medicament of axotrophin or a polypeptide or polynucleotide encoded by or derived from axotrophin or a substance that alters amount or activity thereof in an individual, to boost or reduce an aggressive immune response against an antigen or to alter tolerance of the immune system to an antigen.

5. Use of axotrophin or a polypeptide or polynucleotide encoded by or derived from axotrophin for assaying immune status.

6. A method of manipulating the response of the immune system to a given antigen in an individual the method comprising administering to the individual axotrophin or a polypeptide or polynucleotide encoded by or derived from axotrophin or a substance that enhances the amount or activity of polypeptide expressed directly or indirectly by axotrophin.

6. A method according to claim 5 comprising potentiating or increasing the aggressive response of the immune system of an individual against an antigen, the method comprising administering to the individual a substance that decreases the amount or activity of a polypeptide expressed directly or indirectly by axotrophin.

7. A method for determining immune status of an individual, the method comprising determining the level of expression of axotrophin or a polypeptide or polynucleotide encoded by or derived from axotrophin in a test sample comprising tissue, cells and/or bodily fluid removed or obtained from the individual and comparing the level for the test sample with that of a control sample, wherein a level in the test sample greater than that of the control sample is indicative that the immune status in the individual comprises a tolerant immune response, the immune status in the individual comprises an aggressive immune response.

8. Isolated axotrophin, a polynucleotide or a polypeptide encoded by or derived from axotrophin.

9. A method for producing axotrophin polypeptide comprising growing a culture of cells in a culture medium under conditions permitting expression of the axotrophin polypeptide, and purifying the polypeptide from the culture or from the host cells.

10. A vector for the expression of axotrophin, a polynucleotide sequence or a polypeptide encoded by or derived from axotrophin, the vector containing axotrophin or a polynucleotide sequence encoding axotrophin, a promoter sequence and a termination sequence.

11. A composition containing axotrophin or a polypeptide or polynucleotide encoded by or derived from axotrophin and a pharmaceutically acceptable diluent, carrier or excipient.

12. A method of screening chemical compounds comprising contacting a test sample containing one or more chemical compounds to be screened with a binder selected from axotrophin, a polynucleotide or polypeptide encoded by or derived from axotrophin and fragment of such polynucleotide or polypeptide and determining whether the chemical compound has bound to the binder.

13. A method according to claim 12 for identifying an agent which binds to axotrophin or a polypeptide or polynucleotide encoded by or derived from axotrophin comprising:
(a) contacting an agent with axotrophin or a or polynucleotide polypeptide encoded by or derived from axotrophin;
(b) determining whether the agent binds to the said polynucleotide or polypeptide; and
(c) detecting the formation of a complex, formed between the agent and the said polynucleotide or polypeptide such that if a complex is formed, the agent is detected.

14. A method according to claim 13 in which the compound is contacted with axotrophin or polypeptide or polynucleotide of axotrophin in a cell wherein the complex drives expression of a receptor gene sequence in the cell, and detecting the complex by detecting reporter gene sequence expression.

15. A kit comprising:
i) a polynucleotide or polypeptide probe and/or monoclonal antibodies said probe or antibodies comprising axotrophin or a polypeptide or polynucleotide encoded by or derived from axotrophin, and optionally
ii) quantitative standards for carrying out method according to any one of the preceding claims.

16. A diagnostic method for detecting axotrophin or a polynucleotide or polypeptide encoded by or derived from axotrophin comprising:
(a) contacting a sample to be tested for the presence of a polynucleotide or polypeptide encoded by or derived from axotrophin with a compound that binds to a polynucleotide or polypeptide encoded by or derived from axotrophin;
(b) determining whether the compound binds to a component of the sample; and
(c) detecting the formation of a complex, formed between the agent and the protein or polynucleotide and such that if a complex is formed, the polypeptide or polynucleotide is detected.

17. A diagnostic method for assessing the immune response of an individual comprising obtaining a test sample from the individual, incubating the test sample with one or more of the antibodies to or polynucleotide probes for axotrophin or a polynucleotide or polypeptide encoded or derived from axotrophin and assaying for binding of the polynucleotide probes or antibodies to a component within the test sample.

18. A method according to claim 16 or claim 17 wherein step (c) comprises testing with an ex vivo immune cell population.

19. A method according to claim 16 or claim 17 wherein step (c) comprises testing in vivo.

20. Use of a compound that binds to a polynucleotide or polypeptide encoded by or derived from axotrophin in the manufacture of a medicament for treating an individual and/or ex vivo cell population to modulate immune response to an antigen.


SEQUENCE LISTING (5 sequences)

SEQ ID NO: 1 - DNA, 2728 bp, Homo sapiens
ggtggctggttctgcgccggatccgggagaggggcgggcgccattgtgcttcgctgccgactgcatttcctcagtcacgggcctagaactccaaggagaaaggcggcgaaaaatctttaagaatggagtctaaaccttcaaggattccaagaagaatttctgttcaaccttccagctccttaagtgctaggatgatgtctggaagcagaggaagtagtttaaatgatacctatcactcaagagactcttcatttagattggattctgaatatcagtctacatcagcatcagcatctgcgtcaccatttcaatctgcatggtatagtgaatctgagataactcagggagcacgctcaagatcgcagaaccagcaacgggatcatgattcaaaaagacctaaactttcctgtacaaactgtactacctcagctgggagaaatgttggaaatggtttaaacacattatcagattcatcttggaggcatagtcaagttcctagatcttcatcaatggtacttggatcatttggaacagacttaatgagagagaggagagatttggagagaagaacagattcctctattagtaatcttatggattatagtcaccgaagtggtgatttcacaacttcatcatatgttcaagacagagttccttcatattcacaaggagcaagaccaaaagaaaactcaatgagcactttacagttgaatacatcatccacaaaccaccaattgccttctgaacatcagaccatactaagttctagggattccagaaattctttaagatcaaatttttcttcaagagaatcagaatcttcccgaagcaatacgcagcctggattttcttacagttcaagtagagatgaagccccaatcataagcaattcagaaagggttgtttcatctcaaagaccatttcaagaatcttctgacaatgaaggtaggcggacaacgaggagattgctgtcacgcatagcttctagcatgtcatctacttttttttcacgaagatctagtcaggattccttgaatacaagatcattgaattctgaaaattcttacgtttctccaagaatcttgacagcttcacagtcccgtagtaatgtaccatcagcttctgaagttcccgataatagggcgtctgaagcttctcagggatttcgatttcttaggcgaagatggggtttgtcatctcttagccacaatcatagctctgagtcagattcagaaaattttaaccaagaatctgaaggtagaaatacaggaccatggttatcttcctcacttagaaatagatgcacacctttgttctctagaaggaggcgagagggaagagatgaatcttcaaggatacctacctctgatacatcatctagatctcatatttttagaagagaatcaaatgaagtggttcaccttgaagcacagaatgatcctcttggagctgctgccaacagaccacaagcatctgcagcatcaagcagtgccacaacaggtggctctacatcagattcggctcaaggtggaagaaatacaggaatatcagggattcttcctggttccttattccggtttgcagtccccccagcacttgggagtaatttgaccgacaatgtcatgatcacagtagatattattccttcaggttggaattcagctgatggtaaaagtgataaaactaaaagtgcgccttcaagagatccagaaagattgcagaaaataaaagagagcctccttttagaggactcagaagaagaagaaggtgacttatgtagaatttgtcaaatggcagctgcatcatcatctaatttgctgatagagccatgcaagtgcacaggaagtttgcagtatgtccaccaagactgtatgaaaaagtggttacaggccaaaattaactctggttcttcattagaagctgtaaccacctgtgaactatgtaaagagaagttggagcttaacctggaggattttgatattcatgaactacatagagctcatgcaaatgaacaagctgagtatgagtttatcagctctggtctctacctagtggtgttattgcacttgtgcgaacaaagcttttctgatatgatgggaaatacaaatgaaccaagcacacgtgtccgatttattaaccttgcaagaactcttcaggcacatatggaagatctcgaaacttcagaggatgattccgaagaagacggagaccataacaggacatttgatattgcctaacttcatataagacagatggatgatctgtgaacataagtgtttattaaaaatggcaattaaatataaattacttttgtgggggaatgcctaataaatacattgactatatataaaatgaatatatacatacacatgtatgcctgtatatatatattcattctccagtgttgctgaattaaaattctgctggactttttaacatagcaaatccgatgtttataaactggtaatcaaaaaggttttttcttttaggtgagtgggaaagtattacccttgttttaaatatctaagcaatgcctatcaacccttttttgtgttatgattactgtagtcatatttatgaaaaaaggtttgtgttttactcttgctagtgagaaaagtgggacaaaatatacttttgaaataaaatgctatatggcacctaattattttttcttttaaaatgccttaagttgcagtctcattttgataatcatttgcttccagtgttt

SEQ ID NO: 2 - DNA, 2720 bp, Mus musculus
cgcatccggaggggcggccgccattgtgcttcgtcgccgacttctctgccggtagcccgagagccgagccgagcccagcgaggaaggcggcggcggtgtggctgcggcgagcgcgacactccctgcagcggagtgctcggtggaagagggaaaccttaagaatggagtctaaaccttccaggattccaagaagaatttctgttcaaccctctggctctttaagcactaggatggtgtctggaaacagaggaaccagtttaaatgattcatatcattctagagactcctcctttagactggattctgaatatcagtctgcatcagcatcagcgtgtgcatcaccatgtcagcctgcctggtacagtgagtctgagatacctcagggagcgcgggcacgagcacagacccagcagcgggatcatgactcaaagagacccaagctttcctgtacaaactgtgcatctacctcagctgggaggaacggtgggagtgggttaaatacagtgtcagattcttcttggaggcatagtcaagttcccagatcttcatcaatggtacttggttcatttggaacagacttgatgagagaaaggagagatttggacaggagaagagagtcctccatcagcaatcttatggattataatcaccgaagtggtgatttcacaacttcatcatatgttcaagaaagagttccttcttcatattcacagggagcaagaccaaaagagaatgcagtgagcactttacagttgaattcatcatccaccaatcaccaattgccttctgaccatcagacagtaccaagttctagggactccagtagaagttctttcagatcacatttttctccaagacaatcagaatcttttcgcaacagttcacatcctgcattttcatatttttcaagtagaaatgaaactccaactataagcaattcagaaaggggttcatctcagagaccatatcgagaatcttctgacaatgaaggtaggcgtacaactaggagattgctgtcacggatagcttctagcatgtcatctacttttttctcacgaagatctagtcaagattccttgaatacaagatctttgagttctgaaaattatatttctccgagaaccctgacttcacagtctcggaataatggaacctcctcgtcctctgacgtcagtgagggcagggcagctgaagcatctcagggatttagatttcttaggcgaagatgggggttgtcgtcgctcagccaaaatcatagctctgaaccagaggcagaaaattttaaccaagaatcagaaggtagaaattcaggaccatggttgtcttcttcacttagaaatagatgcacacctttgttctcgagaaggaggcgagagggaagggatgagtcttcaagaatgtctacgtcagatgtaccacctagatctcatattttcagaagagattcaaatgaagtagttcatcttgaagcacagggtgactcccttggggctgctgccaaccgaccacaagcatctggagcgtcaagcagtgctgctgcaggtggctccaccccagagttgcctcagggtggaagaaatccaggactaacagggattcttcctggctccttgttccggtttgcagtcccaccagcactcggcagtaatctggctgacaatgtcatgattactgtagatattatcccttctggttggaattcaactgatgggaaaaatgataaagctaaaagtgcaccttcaagagacccagaaaaacttcagaaaatcaaagaaagcctccttttagaggactctgatgatgaagaagaaggggacttatgtagaatttgtcagatggcagcagcgtcatcatctaatttattgatagagccgtgcaaatgcacagggagcctgcagtacgtccatcaagagtgtatgaaaaagtggttacaagccaaaattaattctggctcttcattagaggctgtgactacctgtgaactctgtaaagagaagttgcaacttaacctggaggattttgatattcatgaactacatagagctcatgcaaatgaacaagctgagtatgagtttatcagctctggtctctacctagttgtcttactgcacttgtgtgaacaaagcttttctgatatgatgggaaatacaattgaaccaagcactcgtgtccgatttattaaccttgcaagaactcttcaggcacatatggaagatctcgaaacttcagaggatgaattctgaagaagatggagaccataagagaatgcttgatattgcctaacttcatttaagaaaaaaaaaaaaaaggatgatctgtgaacatgtttattaaaactggcaattaagtatggataatttcatggggtaatgcctagtagattaattgactatacataaaatgaatatatatatatacatgtataaatgtaaatatatattcattctcaagtattgctgaactgaaattcttgagctggaccctttaacactggccagcgaatctcatgtttataatatgtaatccaagcatttttccttttggtgagtgggaaagcattacccttgtttgaaatatctaaacagtgctcatcaactttcttctttgttgcaattactgtagtcatatttatgggaaaaaaatgtttgtgtattagtctcttgctagtgaaaaaaagtcagataaaatgtccttttgaaataaaatgccaatggcaccta

SEQ ID NO: 3 - protein, 704 aa, Homo sapiens
MESKPSRIPRRISVQPSSSLSARMMSGSRGSSLNDTYHSRDSSFRLDSEYQSTSASASASPFQSAWYSESEITQGARSRSQNQQRDHDSKRPKLSCTNCTTSAGRNVGNGLNTLSDSSWRHSQVPRSSSMVLGSFGTDLMRERRDLERRTDSSISNLMDYSHRSGDFTTSSYVQDRVPSYSQGARPKENSMSTLQLNTSSTNHQLPSEHQTILSSRDSRNSLRSNFSSRESESSRSNTQPGFSYSSSRDEAPIISNSERVVSSQRPFQESSDNEGRRTTRRLLSRIASSMSSTFFSRRSSQDSLNTRSLNSENSYVSPRILTASQSRSNVPSASEVPDNRASEASQGFRFLRRRWGLSSLSHNHSSESDSENFNQESEGRNTGPWLSSSLRNRCTPLFSRRRREGRDESSRIPTSDTSSRSHIFRRESNEVVHLEAQNDPLGAAANRPQASAASSSATTGGSTSDSAQGGRNTGISGILPGSLFRFAVPPALGSNLTDNVMITVDIIPSGWNSADGKSDKTKSAPSRDPERLQKIKESLLLEDSEEEEGDLCRICQMAAASSSNLLIEPCKCTGSLQYVHQDCMKKWLQAKINSGSSLEAVTTCELCKEKLELNLEDFDIHELHRAHANEQAEYEFISSGLYLVVLLHLCEQSFSDMMGNTNEPSTRVRFINLARTLQAHMEDLETSEDDSEEDGDHNRTFDIA

SEQ ID NO: 4 - protein, 693 aa, Mus musculus
MESKPSRIPRRISVQPSGSLSTRMVSGNRGTSLNDSYHSRDSSFRLDSEYQSASASACASPCQPAWYSESEIPQGARARAQTQQRDHDSKRPKLSCTNCASTSAGRNGGSGLNTVSDSSWRHSQVPRSSSMVLGSFGTDLMRERRDLDRRRESSISNLMDYNHRSGDFTTSSYVQERVPSSYSQGARPKENAVSTLQLNSSSTNHQLPSDHQTVPSSRDSSRSSFRSHFSPRQSESFRNSSHPAFSYFSSRNETPTISNSERGSSQRPYRESSDNEGRRTTRRLLSRIASSMSSTFFSRRSSQDSLNTRSLSSENYISPRTLTSQSRNNGTSSSSDVSEGRAAEASQGFRFLRRRWGLSSLSQNHSSEPEAENFNQESEGRNSGPWLSSSLRNRCTPLFSRRRREGRDESSRMSTSDVPPRSHIFRRDSNEVVHLEAQGDSLGAAANRPQASGASSSAAAGGSTPELPQGGRNPGLTGILPGSLFRFAVPPALGSNLADNVMITVDIIPSGWNSTDGKNDKAKSAPSRDPEKLQKIKESLLLEDSDDEEEGDLCRICQMAAASSSNLLIEPCKCTGSLQYVHQECMKKWLQAKINSGSSLEAVTTCELCKEKLQLNLEDFDIHELHRAHANEQAEYEFISSGLYLVVLLHLCEQSFSDMMGNTIEPSTRVRFINLARTLQAHMEDLETSEDEF

SEQ ID NO: 5 - protein, 4 aa, Artificial Sequence
  Description of Artificial Sequence Synthetic peptide
ELKL